(19) United States
(12) Patent Application Publication
(10) Pub. No.: US 2009/0208944 A1
(43) Pub. Date: Aug. 20, 2009

(54) ASSESSING OUTCOMES FOR BREAST CANCER PATIENTS TREATED WITH TAMOXIFEN

(76) Inventors: Matthew P. Goetz, Rochester, MN (US); James N. Ingle, Rochester, MN (US); Richard M. Weinshilboum, Rochester, MN (US); Fergus J. Couch, Rochester, MN (US); Matthew M. Ames, Rochester, MN (US)

Correspondence Address: FISH & RICHARDSON P.C., PO BOX 1022, MINNEAPOLIS, MN, 55440-1022, US

(21) Appl. No.: 12/096,723

(22) PCT Filed: Dec. 8, 2006
(86) PCT No.: PCT/US06/47043

§ 371 Date: Jan. 16, 2009

Related U.S. Application Data
(60) Provisional application No. 60/748,770, filed on Dec. 9, 2005.

(51) Int. Cl. C12Q 1/68

(52) U.S. Cl. 435/6

(57) ABSTRACT

This document provides methods and materials related to assessing the likely outcome for mammals (e.g., humans) with cancer (e.g., breast cancer). For example, methods and materials that involve assessing a breast cancer patient's cytochrome P450, family 2, subfamily D, polypeptide 6 (CYP2D6) genotype to determine the likelihood of the beast cancer patient to experience breast cancer relapse or death are provided. Methods and materials that involve assessing the likelihood that a breast cancer patient being treated with tamoxifen will experience side effects such as hot flashes also are provided.

### BACKGROUND

1. Technical Field

This document relates to methods and materials involved in assessing the outcome of cancer patients treated with tamoxifen.

2. Background Information

In the adjuvant treatment of estrogen receptor (ER) positive breast cancer, hormonal therapy reduces the risk of breast cancer recurrence and decreases mortality. Tamoxifen, one of the most commonly used medications in the adjuvant treatment of ER positive breast cancer, is a selective ER modulator that competes with estrogen for binding to the ER. When administered to women with surgically treated ER positive breast cancer, tamoxifen reduces the risk of recurrence and death when taken for five years.

### SUMMARY

This document provides methods and materials related to assessing the likely outcome for mammals (e.g., humans) with cancer (e.g., breast cancer). For example, this document provides methods and materials that involve assessing a breast cancer patient's cytochrome P450, family 2, subfamily D, polypeptide 6 (CYP2D6) genotype to determine the likelihood of the beast cancer patient to experience breast cancer relapse or death. This document also provides methods and materials that involve assessing the likelihood that a breast cancer patient being treated with tamoxifen will experience side effects such as hot flashes. As described herein, women with the CYP2D6*4/*4 genotype tend to have a higher risk of disease relapse and a lower incidence of hot flashes, relative to women heterozygous or homozygous for the wild-type CYP2D6 allele.

In general, one aspect of this document features a method for assessing the likelihood of cancer relapse. The method comprises, or consists essentially of, determining whether or not a breast cancer patient contains a CYP2D6*4/*4 genotype, where the presence of the genotype indicates that the patient is likely to experience cancer relapse (e.g., breast cancer relapse) or a shorter relapse-free survival than the relapse-free survival for a population of comparable patients lacking the CYP2D6*4/*4 genotype or a shorter disease-free survival than the disease-free survival for a population of comparable patients lacking the CYP2D6*4/*4 genotype, and where the absence of the genotype indicates that the patient is likely to experience a relapse-free survival, a disease-free survival, a longer relapse-free survival than the relapse-free survival for a population of comparable patients having the CYP2D6*4/*4 genotype, or a longer disease-free survival than the disease-free survival for a population of comparable patients having the CYP2D6*4/*4 genotype or is unlikely to experience cancer relapse (e.g., breast cancer relapse). The patient can contain the CYP2D6*4/*4 genotype, and the method can comprise classifying the patient as being likely to experience cancer relapse. The patient can lack the CYP2D6*4/*4 genotype, and the method can comprise classifying the patient as being likely to experience a relapse-free survival or disease-free survival. The determining step can comprise using PCR. The determining step can comprise obtaining nucleic acid from a tumor sample of the patient. The determining step can comprise obtaining nucleic acid from a non-tumor sample of the patient. The patient can be a tamoxifen-treated patient.

In another embodiment, this document features a method for assessing the likelihood of a breast cancer patient to have hot flashes when treated with tamoxifen. The method comprises, or consists essentially of, determining whether or not the breast cancer patient contains a CYP2D6*4/*4 genotype, where the presence of the genotype indicates that the patient is unlikely to experience hot flashes when treated with tamoxifen, and where the absence of the genotype indicates that the patient is likely to experience hot flashes when treated with tamoxifen. The patient can contain the CYP2D6*4/*4 genotype, and the method can comprise classifying the patient as being unlikely to experience hot flashes when treated with tamoxifen. The patient can lack the CYP2D6*4/*4 genotype, and the method can comprise classifying the patient as being likely to experience hot flashes when treated with tamoxifen. The determining step can comprise using PCR. The determining step can comprise obtaining nucleic acid from a tumor sample of the patient. The determining step can comprise obtaining nucleic acid from a non-tumor sample of the patient.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is a schematic diagram of tamoxifen transformation pathways listing the primary CYP enzymes involved. The relative contribution of each pathway to the overall oxidation of tamoxifen is shown by the thickness of the arrow, and the principal P450 isoforms responsible are highlighted in larger fonts.

FIG. 2 is a graph plotting Kaplan-Meier Estimates of relapse-free survival for patients with the CYP2D6*4 genotype.

FIG. 3 is a graph plotting Kaplan-Meier Estimates of disease-free survival for patients with the CYP2D6*4 genotype.

FIG. 4 is a graph plotting Kaplan-Meier Estimates of overall survival for patients with the CYP2D6*4 genotype.

FIG. 5 contains graphs plotting Kaplan-Meier Estimates of time to breast cancer recurrence (Panel A), relapse-free survival (Panel B), disease-free survival (Panel C), and overall survival (Panel D) based on CYP2D6 metabolism (extensive vs. decreased).

FIG. 6 contains graphs plotting Kaplan-Meier Estimates of time to breast cancer relapse (Panel A), relapse-free survival (Panel B), disease-free survival (Panel C), and overall survival (Panel D) based on metabolizer status (extensive, intermediate, or poor).

FIG. 7 is a graph plotting smoothed hazard rates for relapse-free survival comparing patients with extensive vs. decreased CYP2D6 metabolism.

### DETAILED DESCRIPTION

This document provides methods and materials related to assessing the likely outcome for mammals (e.g., humans) with cancer (e.g., breast cancer). For example, this document provides methods and materials that involve assessing a breast cancer patient's CYP2D6 genotype to determine the likelihood of the beast cancer patient to experience breast cancer relapse or death. A breast cancer patient can be a breast cancer patient treated with tamoxifen or can be a breast cancer patient not receiving a cancer treatment. As described herein, women with a CYP2D6*4/*4 genotype tend to have a higher risk of disease relapse than women lacking a CYP2D6*4/*4 genotype. Any method can be used to determine whether or not a mammal has a CYP2D6*4/*4 genotype. For example, standard PCR and sequencing techniques can be used to determine the presence or absence of a CYP2D6*4/*4 genotype.

This document also provides methods and materials that involve assessing the likelihood that a breast cancer patient being treated with tamoxifen will experience side effects such as hot flashes. As described herein, women with a CYP2D6*4/*4 genotype tend to have a lower incidence of hot flashes when treated with tamoxifen than women lacking a CYP2D6*4/*4 genotype.

### EXAMPLES

### Example 1

### Assessing Breast Cancer Outcomes

### Methods and Materials

Patients. The North Central Cancer Treatment Group (NCCTG) conducted a randomized phase III clinical trial in postmenopausal women with resected ER positive breast cancer to assess the value of adding one year of fluoxymesterone to 5 years of tamoxifen adjuvant therapy (NCCTG 89-30-52; Ingle et al., Proc. Am. Soc. Clin. Oncol., 19:86a (2000)). No women received adjuvant chemotherapy.

The details of the clinical trial including the eligibility requirements were as follows. Postmenopausal women with node-negative disease were required to have a stage T₁c or T₂N₀M₀ and could be any age, whereas women with node-positive disease were required to be at least 65 years of age with a tumor stage T₁ (any N) or T₂N₁M₀. A woman was classified as postmenopausal if one of the following held: 1) her last menstrual cycle was >12 months prior to diagnosis, 2) her last menstrual cycle was 4-12 months prior to diagnosis and her follicle-stimulating hormone (FSH) level in the postmenopausal range, 3) she had had a bilateral oophorectomy at least 2 months prior to diagnosis, or 4) she had had a hysterectomy without oophorectomy and was either >60 years old or her FSH was in the postmenopausal range. Patients were surgically treated with either a modified radical mastectomy or breast conservative therapy including lumpectomy, axillary nodal dissection, and radiation therapy. The axillary dissection must have involved at least levels I and II, and the examination of at least 6 axillary nodes. Patients who underwent lumpectomy must have had a primary tumor no larger than 5 cm, and the surgical margins must have been microscopically free of tumor. Postlumpectomy radiation therapy consisted of a total cumulative breast dose of 5040 cGy in 28 fractions, and those with axillary nodal involvement also received radiation to the axilla and supraclavicular regions. Patients were classified as ER positive if ≧10 fmol/mg cytosol protein or positive by an immunohistochemical assay. All patients were randomized within 6 weeks of definite surgery.

Contraindications for entry onto protocol included pectoral fascia invasion, bilateral or previous breast cancer, other cancer with exception of resected non-melanoma skin cancer or adequately treated carcinoma in-situ of the uterine cervix unless disease-free for at least 5 years, white blood cell count less than 3000/μL, platelet count less than 100,000/μL, total bilirubin or SGOT over 1.5 times the institutional upper limit of normal (IULN), creatinine >2 times the IULN, warfarin therapy, and prior systemic therapy for breast cancer with the exception of tamoxifen administered within 14 days of randomization.

A total of 541 patients were randomized to either oral tamoxifen, 20 mg daily for 5 years (256 eligible) or tamoxifen, 20 mg daily for 5 years plus oral fluoxymesterone, 10 mg twice daily for one year (258 eligible). Patients were stratified based on axillary lymph node status (0, 1-3, 4-9 vs. 10 or more), age (<65 years vs. >65), primary tumor size (<3 cm vs. >3 cm), ER status (10-49 fmol vs. 50 fmol or greater vs. positive by immunohistochemical assay) and local therapy (mastectomy vs. breast conservation therapy).

Clinical evaluations including history, physical examination, blood and chemistry groups, chest x-ray, and toxicity assessments were performed every 4 months for the first year, every 6 months years 2-5, and then yearly. Mammograms and pelvic examinations were performed annually. Toxicities were graded using the NCI Common Toxicity Criteria version 1.0 and the NCCTG supplement in which hot flashes were graded as O-none or no change, 1-mild, 2-moderate, or 3-severe.

Sample Preparation. Utilizing paraffin-embedded tissue blocks from the women enrolled to the tamoxifen only arm, three sections (10-μm thick) and one hematoxylin and eosin slide were prepared from each block, and a 1 cm area of high tumor cellularity was identified, microdissected, deparaffinized, and DNA extracted using the method described elsewhere (Rae et al., Pharmacogenetics, 13:501-7 (2003)). In those living women that supplied a buccal sample, DNA was extracted using a QIAamp DNA Mini kit (Qiagen, Valencia, Calif.) per instructions.

Assay methods. Patient samples (both tumor and buccal) were genotyped for CYP2D6*4 (1846G>A), and CYP2D6*6 (1707T>del) polymorphisms as described elsewhere (Jin et al., J. Natl. Cancer Inst., 97:30-9 (2005)) using the Applied Biosystems' Taqman® Allelic Discrimination Assay (Foster City, Calif.) according to the manufacturer's instructions. Briefly, 10 ng DNA was added to a 5 μL reaction containing forward and reverse primers along with 2 allele specific labeled probes (one wild-type and one variant allele specific). The PCR and fluorescence measurements were performed using the ABI Prism 770 sequence detection system. Additional CYP2D6 alleles less common in the Caucasian population (*3 and *5) were not analyzed because of inadequate amounts of DNA were available for testing.

Additionally, patient samples (both tumor and buccal) were genotyped for the CYP3A5*3 polymorphism (6986 G>A) using the method described elsewhere (Hustert et al., Pharmacogenetics, 11:773-9 (2001)) with minor modifications. Briefly, a 280 base pair (bp) product was amplified using an initial 10 minute incubation at 94° C., followed by 45 cycles of: 1 min at 94° C., 1 min at 67° C., and 1 min at 72° C. with a final 5 minute extension at 72° C. Amplification was confirmed on a 2% agarose gel, and the samples were sequenced by the dye terminator method in both the forward and reverse directions. Sequencing results were manually evaluated at the SNP location.

Study Design and End Points. One objective of this study was to determine the relationship between genotype and relapse-free time, disease-free survival, and overall survival. Another objective was to determine whether the incidence of hot flashes differed with respect to genotype.

Relapse-free (RF) time was defined as the time from randomization to documentation of a breast event where a breast event is any recurrence (local, regional or distant) of breast cancer or the documentation of contralateral breast cancer (including ductal carcinoma in situ). When estimating the distribution of RF time, patients who developed a non-breast second primary cancer (other than squamous or basal cell carcinoma of the skin, carcinoma in situ of the cervix, or lobular carcinoma in situ of the breast) prior to the diagnosis of a breast event were censored on the day their second primary was diagnosed. Patients (alive or dead) without a breast recurrence, contralateral breast cancer or a second non-breast primary cancer were censored at the date of their last disease evaluation.

Disease-free survival (DFS) was defined as the time from randomization to documentation of the first of the following events: any recurrence (local, regional or distant) of breast cancer, the documentation of contralateral breast cancer, or death due to any cause. Patients who were alive without a breast recurrence, contralateral breast cancer or a second non-breast primary cancer were censored at the date of their last disease evaluation. Patients who developed a second primary (prior to breast recurrence or a contralateral breast cancer) were censored on the date the second primary was diagnosed.

Overall survival (OS) was estimated as the time from registration to death due to any cause.

The distributions of RF time, DFS, and OS were estimated overall using the Kaplan-Meier method. For each patient and pathologic factor and each clinical outcome, the following approach was used to assess the strength of the relationship between the factor and the outcome and to assess the proportional hazard assumption. A log rank test was used to assess the association between the factor and the outcome of interest. To examine whether the proportional hazards assumption was appropriate, a plot of the log of the stratum-specific cumulative hazard functions of that factor (determined using Kaplan Meier estimates) against time was constructed and examined for lack of parallelism. Also, a Cox model with the factor of interest and the interaction term composed of the factor with log(time) was fit to the data and then the significance of the interaction term was assessed to evaluate whether the hazard depended upon time.

Cox multivariate modeling was then used to determine which subset of patient and pathologic characteristics from among age (<65 years vs. >65), extent of surgery (mastectomy vs. breast conservation therapy), primary tumor size (<3 cm vs. >3 cm), axillary lymph node status (positive vs. negative), and ER status (10-49 fmol vs. 50 fmol or greater vs. positive by immunohistochemical assay) was significantly associated with each clinical endpoint. Model assumptions and adequacy of fit were examined using residual plots [such as: dfbeta statistic (a transform of the score residual) versus patient rank order of study entry, Martingale residuals versus linear predictor scores, and deviance residual versus linear predictor scores].

The potential prognostic value of each genotype in terms of RF time, DFS, and OS was expressed using three categories: no variant alleles, one variant allele, and two variant alleles. The log-rank test and the generalized Wilcoxon test were then used to assess whether RF time, DFS, or OS differed with respect to genotype. For each endpoint and each genotype, the Cox model that was previously found to provide the best fit from among those containing a subset of patient and pathologic characteristics was expanded to include the genotype represented in terms of 2 indicator variables. The likelihood ratio tests were then used to ascertain whether one or both indicator variables made a significant contribution to the model. For all CYP2D6*4 analyses, the “wild-type” allele refers to the absence of the *4 allele. Hot flashes were graded using the NCCTG supplement to the NCI common toxicity criteria (version 1) as follows: O-none or no change, I-mild, 2-moderate, or 3-severe. The Wilcoxon rank sum test was used to assess whether the severity of hot flashes (0-3) differed with respect to genotype, and the one-sided Fisher's exact test was used to assess whether the proportion of women with moderate or severe hot flashes was smaller for those with the CYP2D6*4/*4 genotype than those without the CYP2D6*4/*4 genotype.

### Results

Characteristics of the Patients. Of the 256 women enrolled to the tamoxifen only arm, 213 paraffin-embedded tumor and 10 normal tissue blocks were available for DNA extraction. Table 1 presents the pre-registration characteristics of the 256 eligible patients randomized to the tamoxifen only arm that did and did not have a specimen from their primary surgery available for genotyping. The overall patient characteristics were similar, although a higher percentage of patients with available tissue had a tumor size greater than 3 cm (22%) compared with the group without tissue available (15%).

TABLE 1
Pre-registration characteristics of the patients randomized to the
tamoxifen arm that did and did not have paraffin embedded tumor
tissue available from their primary breast surgery. Additionally, the
patient characteristics of the CYP2D6*4/*4 group are shown.

| | | Women | |
|---|---|---|---|
| | Women with | without | CYP2D6*4/*4 |
| | paraffin tissue | paraffin tissue | genotype |
| | (n = 223) | (n = 33) | (n = 13) |
| Operative procedure | | | |
| Mastectomy | 83% | 73% | 92% |
| Breast conservation | 17% | 27% | 8% |
| Prior Hysterectomy | 42% | 45% | 40% |
| Prior BSO | 25% | 21% | 23% |
| Exogenous Estrogens | 16% | 15% | 0% |
| Number of Positive | | | |
| Nodes | | | |
| 0 | 62% | 64% | 31% |
| 1-3 | 26% | 15% | 54% |
| 4-9 | 7% | 15% | 8% |
| 10+ | 4% | 6% | 8% |
| Tumor Size ≧3 cm | 22% | 15% | 38% |
| ER status | | | |
| 10-49 fmols | 21% | 15% | 15% |
| ≧50 fmols | 67% | 61% | 62% |
| Positive | 12% | 24% | 23% |
| Median age (range) | 68 (42-87) | 69 (48-83) | 73 (56-87) |
| Caucasian | 92% | 91% | 100% |

For the group of 223 patients whose paraffin sample was available, the first documented event was as follows: local, regional or distant breast recurrence (43 patients), contralateral breast cancer (12 patients), a second non-breast primary cancer (16 patients), and death without a breast recurrence or second primary cancer (40 patients). At last follow-up, 112 women are alive without evidence of a breast event or second primary, 25 are alive following a breast event or second primary cancer, 33 died with disease recurrence, 13 died having developed a second primary cancer, 32 died of other causes, and 8 died of unknown causes. The Kaplan-Meier estimates for the 10 year RF time, DFS and OS were as follows: 75.0% (95% CI: 69.1-81.4%), 61.0% (54.7-68.0%), and 68.4% (62.5-74.9%). The median length of follow-up among the 137 patients still alive was 11.4 years (range: 5.7-14.1 yrs).

For the clinical endpoints of RF time, DFS, and OS, Cox modeling demonstrated that positive nodes and tumor size greater than 3 cm were significantly associated with decreased RF time, DFS, and OS.

Genotype and allele frequency. The CYP2D6 (*4, and *6) and CYP3A5 (*3) alleles were successfully amplified in 190, 194, and 205 patients, respectively, and their allelic frequencies are shown in Table 2. No CYP2D6 (*6) variants were detected. The genotype and allelic frequencies for each variant were similar to published reports in a predominantly Caucasian population.

TABLE 2
Genotype and allele frequencies (q) for CYP2D6 (*4), and CYP3A5*3.
No CYP2D6 (*6) variants were observed.

| CYP2D6 (*4) n = 190 | | q = 0.17 |
|---|---|---|
| Wt/Wt | 137 (72.1%) | |
| Wt/*4 | 40 (21.1%) | |
| *4/*4 | 13 (6.8%) | |
| CYP3A5 (*3) n = 205 | | q = 0.088 |
| A/A | 6 (2.9%) | |
| A/G | 24 (11.7%) | |
| G/G | 175 (85.4%) | |

### Clinical Outcome by Genotype.

### A. CYP2D6*4

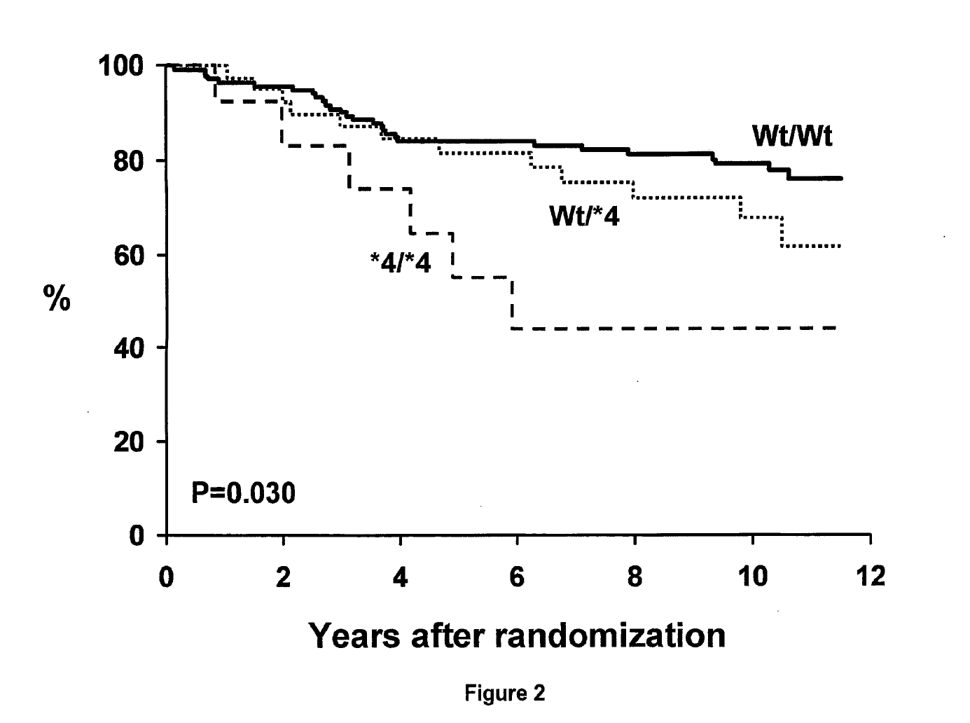

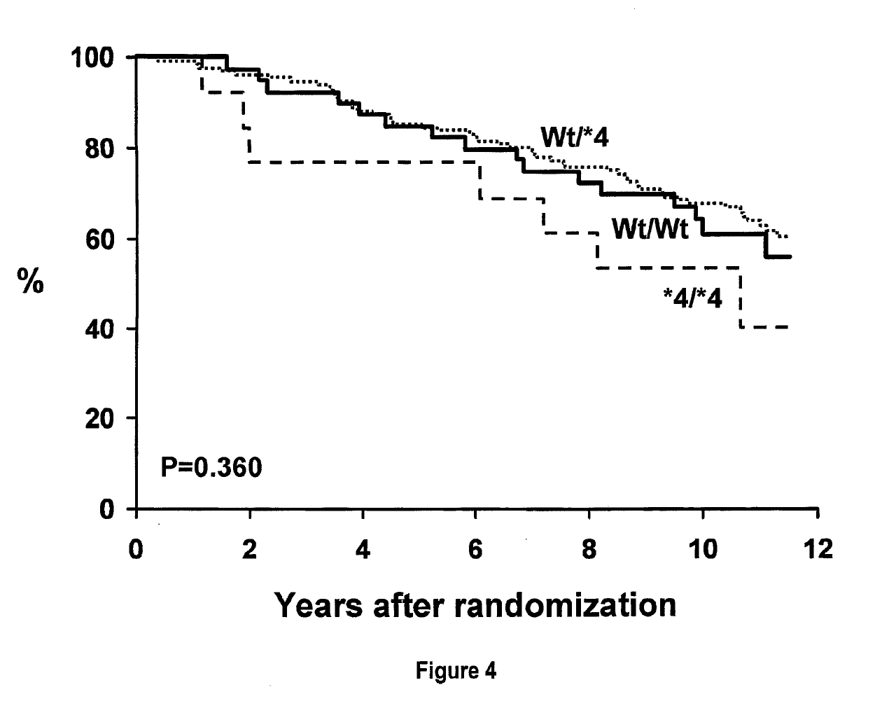

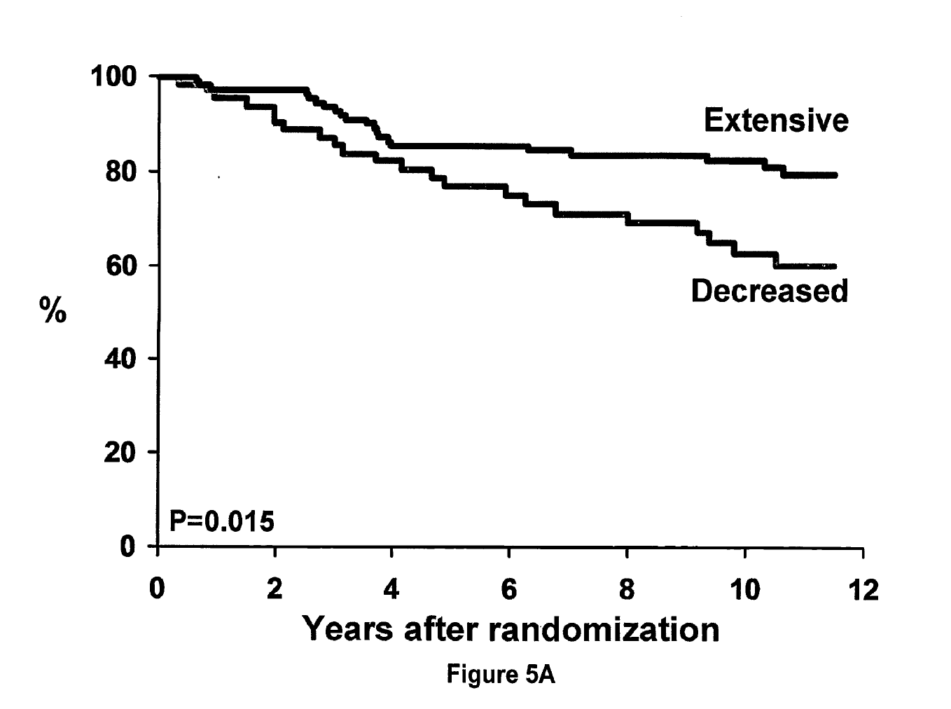

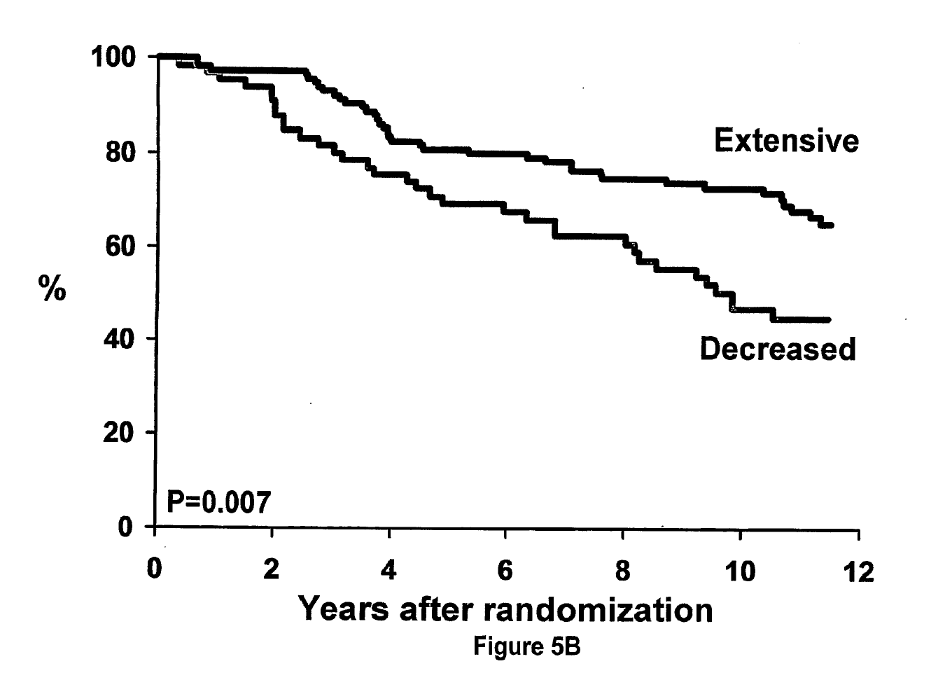

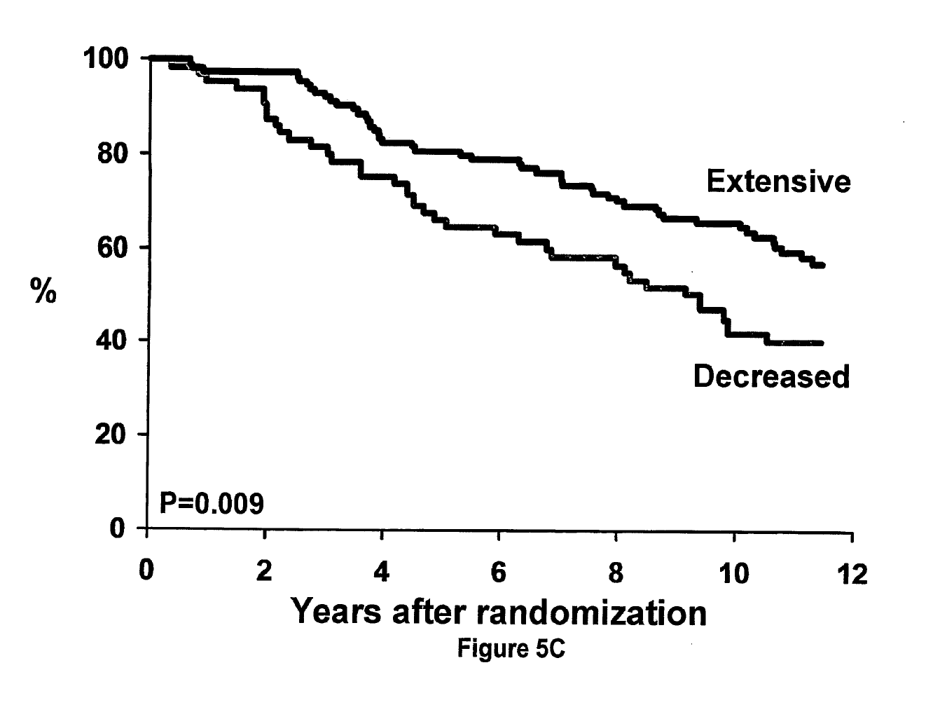

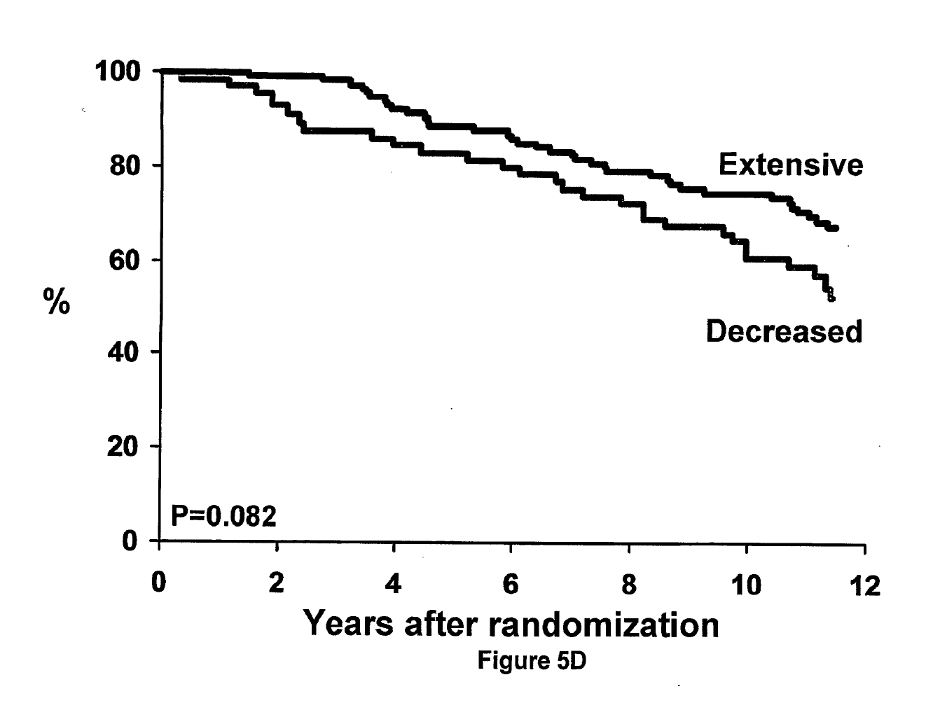

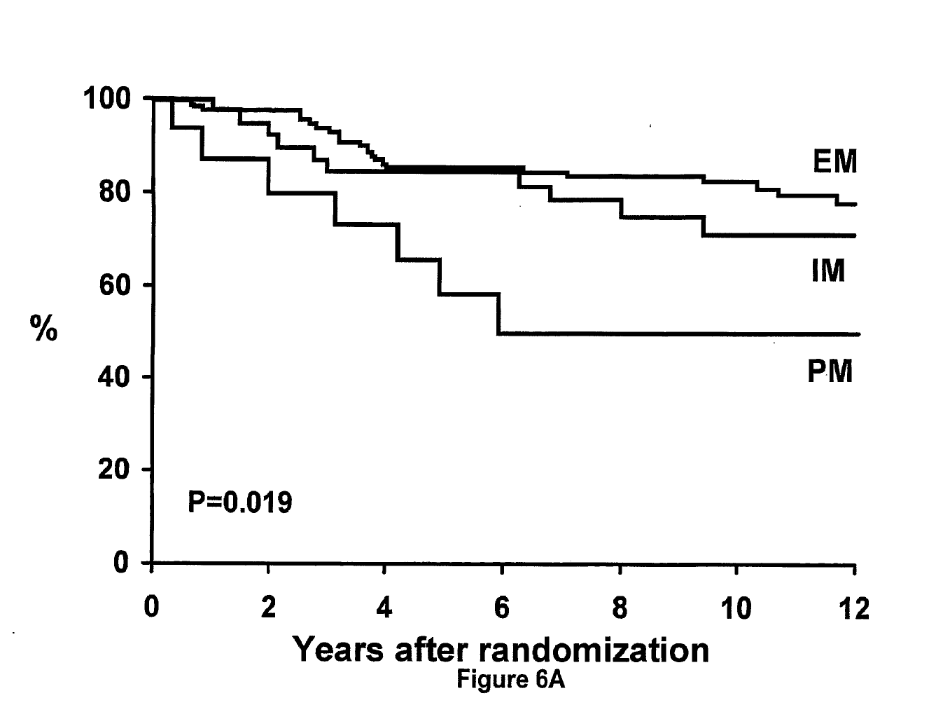

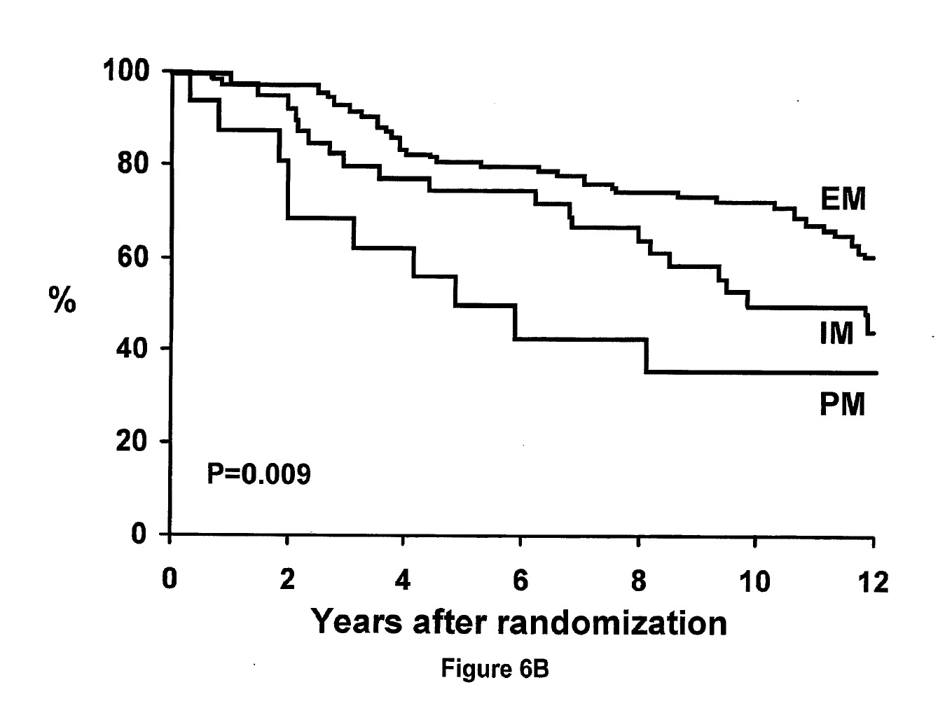

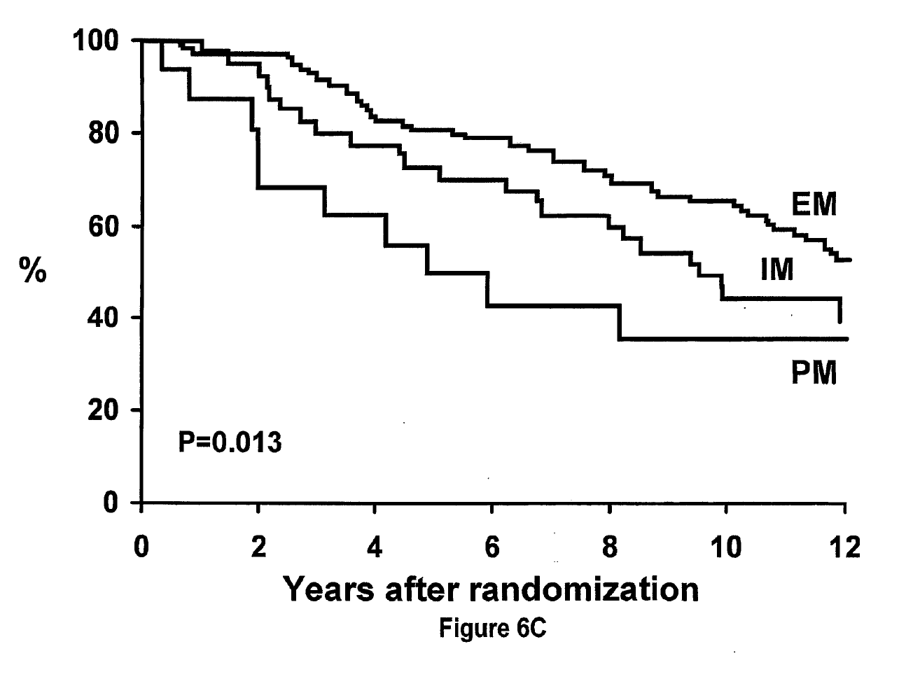

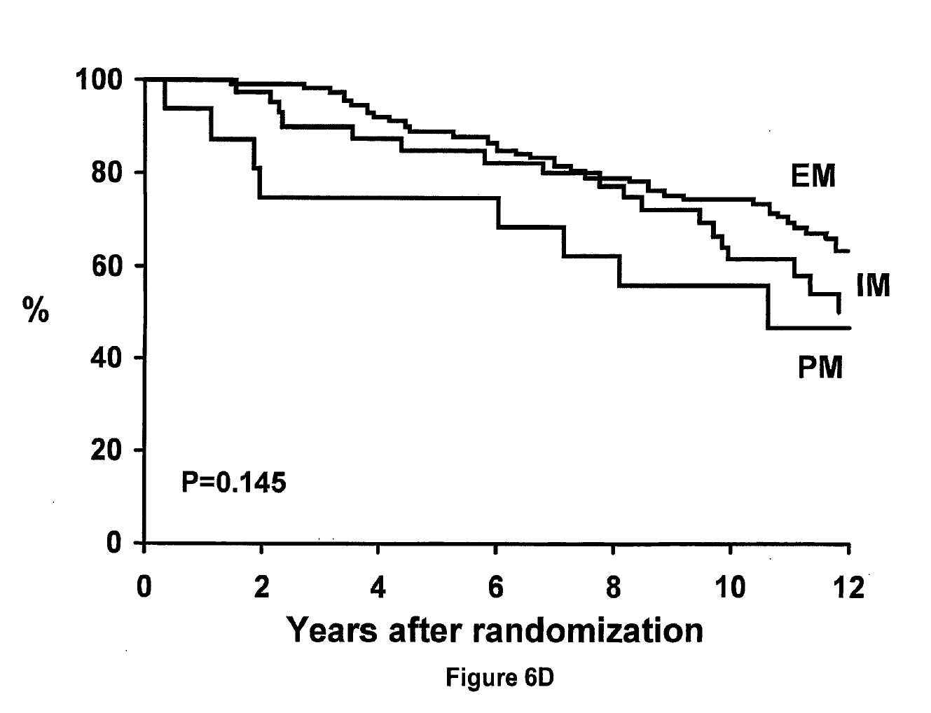

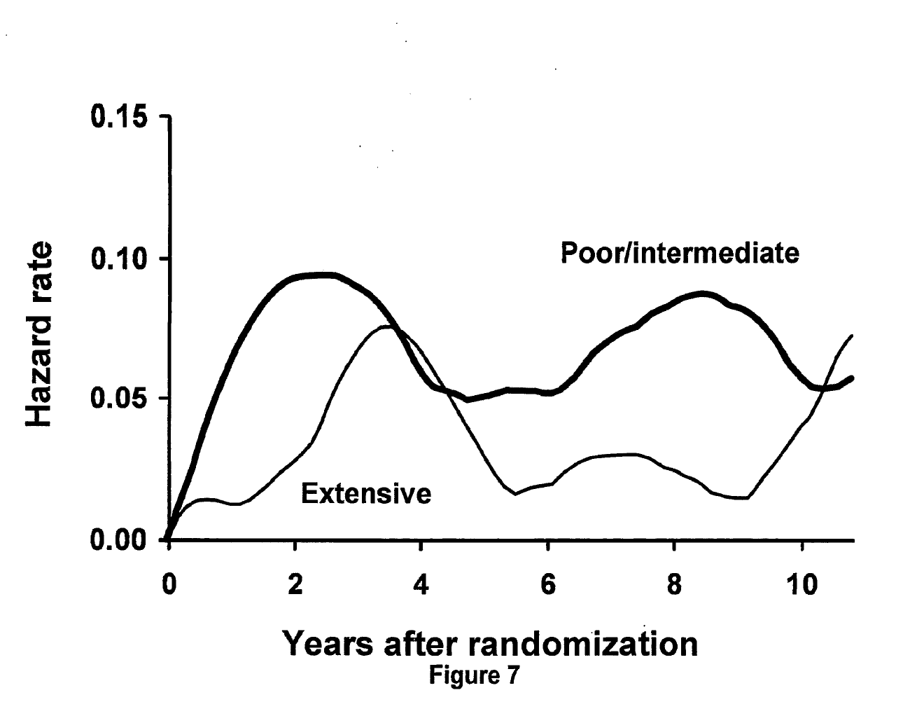

Patient characteristics for women with the CYP2D6*4/*4 genotype are presented in Table 1. A greater proportion of women with the CYP2D6*4/*4 genotype had a node-positive disease relative to that of the entire group. Women with the CYP2D6*4/*4 genotype (n=13) had significantly worse RF time and DFS, but not OS, compared with either the *4/wt (n=40) or the wt/wt genotype (n=137) (log rank p=0.030, p=0.020, and p=0.360, respectively) (FIGS. 2-4). Cox proportional hazard modeling demonstrated that nodal status and tumor size were significantly associated with RF time, DFS, and OS. Once nodal status and tumor size were accounted for, women with the CYP2D6*4/*4 genotype still tended to have worse RF time and DFS, but not OS, compared with patients with one or no variant alleles. Table 3 shows the unadjusted and adjusted hazard ratios comparing patients with the CYP2D6*4/*4 genotype with the wt/wt or *4/wt genotypes.

TABLE 3
Unadjusted and adjusted hazard ratios and corresponding 95%
confidence intervals and p values comparing patients with the
CYP2D6*4/*4 genotype with the WT/WT or *4/WT genotypes.
Hazard Ratios for CYP2D6*4: *4/*4 relative to*4/WT and WT/WT

| | Unadjusted | | Adjusted* | |
|---|---|---|---|---|
| | Hazard Ratio | | Hazard Ratio | P |
| | (95% CI) | P value | (95% CI) | value |
| Relapse-free time | 2.71 (1.15-6.41) | 0.023 | 1.85 (0.76-4.52) | 0.176 |
| Disease-free | 2.44 (1.22-4.90) | 0.012 | 1.86 (0.91-3.82) | 0.089 |
| Survival | | | | |
| Overall Survival | 1.73 (0.79-3.76) | 0.169 | 1.12 (050-2.50) | 0.780 |

*A Cox model including nodal status and tumor size was used to estimate the adjusted hazard ratios

### B. CYP3A5 (*3)

Neither RF time, DFS, nor OS was found to differ in terms of CYP3A5*3 genotype (A/A vs. A/G vs. GIG: log-rank p-value=0.854, 0.937, and 0.950 respectively).

Hot Flashes. Among the 223 patients, a total of 61% (n=136) reported having hot flashes with 40% (n=90) reporting mild (grade 1), 15% (n=34) reporting moderate (grade 2), and 5% (n=12) reporting severe (grade 3) hot flashes. There were no differences in hot flash severity with respect to CYP3A5. However, for CYP2D6*4, none (0/13) of the women with the CYP2D6*4/*4 genotype had moderate or severe hot flashes compared with 20% (36/177) for patients with either the *4/WT or WT/WT genotypes (one-sided p=0.06) (Table 4).

TABLE 4
Incidence of moderate (grade 2) or severe (grade 3) hot flashes within
genotype subgroups.

| | % of patients who developed |
|---|---|
| Genotype | moderate or severe hot flashes |
| CYP3A5 (*3) [n = 205] | |
| G/G | 37/175 (21%) |
| G/A | 6/24 (25%) |
| A/A | 1/6 (17%) |
| CYP2D6 (*4) [n = 190] | |
| *4/*4 | 0/13 (0%) |
| *4/Wt | 9/40 (23%) |
| Wt/Wt | 27/137 (20%) |

Tumor and germline genotype concordance. A total of 17 living women submitted a buccal specimen. For CYP2D6*4, there were 15 patients with concomitant tumor and buccal genotype, and the concordance between tumor and buccal genotype was 100% (15/15). Similarly for CYP3A5*3, there were 13 patients with concomitant tumor and buccal genotype and the concordance rate was 100% (13/13).

The results provided herein demonstrate that women homozygous for the most common allele associated with the CYP2D6 poor metabolizer (PM) phenotype, CYP2D6*4, tend to have worse RF time (HR 1.85, p=0.176) and DFS (HR 1.86, p=0.089), once nodal status and tumor size were accounted for. The biologic importance of a CYP2D6 genotype was further supported by the finding that none of the women with the *4/*4 genotype experienced moderate or severe hot flashes compared with 20% of the women with either the *4/WT or WT/WT genotypes. By obtaining germline CYP2D6*4 genotype from living patients, 100% concordance between tumor and buccal genotype was demonstrated, suggesting that tumor CYP2D6*4 genotype is an accurate means by which to obtain CYP2D6*4 germline status.

In addition, the results provided herein suggest that CYP2D6 genetic variation is a determinant of tamoxifen effect and that lower or absent CYP2D6 activity may increase the risk of tamoxifen treatment failure. These findings can be confirmed in a larger prospective tamoxifen study in which a greater number of alleles corresponding to the CYP2D6 metabolizer status are evaluated.

### Example 2

### Assessing the Effect of CYP2D6 Metabolism on Breast Cancer Outcomes

### Methods and Materials

Patients. The North Central Cancer Treatment Group (NCCTG) conducted a randomized phase III clinical trial in postmenopausal women with resected ER positive breast cancer to assess the value of adding the androgen fluoxymesterone, for one year, to the standard five years of tamoxifen adjuvant therapy (NCCTG 89-30-52). For this trial, ER-positive was defined as ≧10 fmol/mg cytosol protein by a standard biochemical assay or positive by an immunohistochemical assay. The details of the clinical trial, including the eligibility requirements, are described elsewhere (Ingle et al., Breast Cancer Res Treat. (2006)).

The associations of CYP3A5 (*3) and CYP2D6 genetic variation (*4 and *6) with the outcomes of breast cancer relapse and death were determined using the 256 eligible patients randomized to the tamoxifen-only arm (Goetz et al., J Clin Oncol., 23(36):9312-9318 (2005)). Paraffin-embedded tissue blocks were available in 223 patients and CYP2D6*4 genotype was determined in 190 patients (Goetz et al., J Clin Oncol., 23(36):9312-9318 (2005)). Evaluation of the role of CYP2D6 inhibitors in patients randomized to the tamoxifen only arm is described below.

Chart Review. Patient charts were reviewed at each randomizing site to determine whether inhibitors of the CYP2D6 enzyme system were co-administered during the five years that tamoxifen was prescribed. Potent inhibitors were: fluoxetine (Jin et al., J Natl Cancer Inst., 97(1):30-39 (2005); Crewe et al., Br J Clin Pharmacol., 34(3):262-265 (1992)) and paroxetine (Jin et al., J Natl Cancer Inst., 97(1):30-39 (2005); Crewe et al., Br J Clin Pharmacol., 34(3):262-265 (1992)). Moderate inhibitors were: sertraline (Jin et al., J Natl Cancer Inst., 97(1):30-39 (2005); Crewe et al., Br J Clin Pharmacol., 34(3):262-265 (1992)), cimetidine (Martinez et al., Clin Pharmacol Ther., 65(4):369-376 (1999)), amiodarone (Funck-Brentanoet al., Clin Pharmacol Ther., 50(3):259-266 (1991); Ohyama et al., Br J Clin Pharmacol., 49(3):244-253 (2000)), doxepin (Szewczuk-Boguslawska et al., Pol J Pharmacol., 56(4):491-494 (2004)), ticlopidine (Ko et al., Br J Clin Pharmacol., 49(4):343-351 (2000)) and haloperidol (Shin et al., Br J Clin Pharmacol., 51(1):45-52 (2001)). The length of time patients were co-prescribed CYP2D6 inhibitors and tamoxifen was recorded as follows: less than 1 year, 1-2 years, 2-3 years, 3-4 years and 4-5 years.

CYP2D6 Metabolizer Status. Using the available in vivo data regarding the combined effect of CYP2D6 genotype and CYP2D6 inhibitors on endoxifen plasma concentrations (Jin et al., J Natl Cancer Inst., 97(1):30-39 (2005)), the impact of CYP2D6 metabolism was examined by assessing CYP2D6*4 genotype and by documenting the co-administration of a CYP2D6 inhibitor during the five years that tamoxifen was prescribed. Extensive metabolizers were women without a CYP2D6*4 allele (Wt/Wt) and who were not prescribed a CYP2D6 inhibitor. Patients with decreased CYP2D6 metabolism were women who either carried either one or two *4 alleles or women with any genotype who were co-prescribed a CYP2D6 inhibitor. Patients with decreased metabolism were classified further based on the potency of the CYP2D6 inhibitor and its effect on endoxifen levels (Jin et al., J Natl Cancer Inst., 97(1):30-39 (2005)). In this analysis, intermediate metabolizers (IM) were defined as 1) patients heterozygous for the *4 allele (*4/Wt) without co-prescription of a CYP2D6 inhibitor, or 2) Wt/Wt genotype with co-administration of a weak/moderate inhibitor. Poor metabolizers (PM) were defined as women 1) homozygous for the *4 allele (*4/*4), 2)*4/Wt and co-administration of a moderate or potent inhibitor, or 3) Wt/Wt and co-administration of a potent inhibitor.

Study Design and End Points. The primary objectives of the study were to determine the effect of CYP2D6 metabolizer status on the outcomes of time to breast cancer recurrence (TTBR), relapse-free survival (RFS), disease-free survival (DFS) and overall survival (OS).

TTBR was defined as the time from randomization to documentation of a breast event where a breast event is any recurrence (local, regional or distant) of breast cancer or the documentation of contralateral breast cancer (including ductal carcinoma in situ). When estimating TTBR, patients who developed a non-breast second primary cancer (other than squamous or basal cell carcinoma of the skin, carcinoma in situ of the cervix, or lobular carcinoma in situ of the breast) prior to the diagnosis of a breast event were censored on the day their second primary was diagnosed. Patients without a breast recurrence, contralateral breast cancer or a second non-breast primary cancer were censored at the date of their last disease evaluation.

RFS was defined as the time from randomization to documentation of the first of the following events: any recurrence (local, regional or distant) of breast cancer, a contralateral breast cancer or death. When estimating the distribution of RFS, patients who developed a non-breast second primary cancer (other than squamous or basal cell carcinoma of the skin, carcinoma in situ of the cervix, or lobular carcinoma in situ of the breast) prior to the diagnosis of a breast event were censored on the day their second primary was diagnosed. Patients who were alive without a breast recurrence, contralateral breast cancer or a second non-breast primary cancer were censored at the date of their last disease evaluation.

DFS was defined as the time from randomization to documentation of the first of the following events: any recurrence (local, regional or distant) of breast cancer, a contralateral breast cancer, a second primary cancer, or death due to any cause. Patients who were alive without any of these events were censored at the date of their last disease evaluation.

OS was estimated as the time from registration to death due to any cause.

The overall distributions of TTBR, RFS, DFS, and OS were estimated using the Kaplan-Meier method. Log rank tests and univariate Cox proportional hazard models were used to assess whether the endpoint differed with respect to any one of the following factors: age 65 years or greater (yes vs. no), extent of surgery (mastectomy vs. breast conserving), ER status as recorded at time of entry into the trial (10-49 fmols vs. ≧50 fmols vs. positive by immunohistochemistry), positive nodes (yes/no), tumor size 3 cm or greater (yes vs. no), Nottingham grade (3 vs. 1 or 2), HER2 expression (3+ vs. 0, 1+, or 2+), and prior exposure to exogenous estrogens (yes vs. no). For each clinical outcome, multivariate Cox proportional hazard modeling was performed to obtain a subset of the potential prognostic factors which provided an adequate fit to the data. Residual plots were examined. Cox multivariate modeling was then used to determine whether decreased metabolism (yes vs. no) made a significant contribution to the previous established model for that clinical endpoint.

The impact of metabolizer status (extensive, intermediate or poor) on RFS, DFS, and OS was further assessed using univariate Cox's models. The RFS hazard function was estimated using a kernel based approach with the global bandwidth selection algorithm and boundary kernel formulation described elsewhere (Muller and Wang, Biometrics, 50(1):61-76 (1994); Hess et al., Stat Med., 18(22):3075-3088 (1999)).

### Results

CYP2D6*4 genotype was obtained in 190 of the 256 eligible patients enrolled where CYP2D6*4/*4 genotype frequency was 6.8% (Goetz et al., J Clin Oncol., 23(36):9312-9318 (2005)). Medical charts were available for review of 225 patients. Thirteen patients (6%) were co-prescribed a CYP2D6 inhibitor (potent (n=3), moderate (n=10)) during the five years of tamoxifen administration. The median duration of co-administration of tamoxifen and the CYP2D6 inhibitor was 2-3 years in 11 patients. In two patients, the duration of therapy was unknown. Metabolizer status (extensive or decreased) was determined in 180/256 eligible patients using CYP2D6*4 genotype and CYP2D6 inhibitor data (Table 1). For the remaining 76 patients, metabolizer status was not determined because either genotype or medication history was unknown.

TABLE 1
Metabolizer status according to CYP2D6*4 genotype and co-
administration of a CYP2D6 inhibitor

| CYP2D6 | Metabolizer | | Moderate | Potent | |
|---|---|---|---|---|---|
| metabolism | status | Genotype | inhibitor | inhibitor | N = 180 |
| Extensive | Extensive | Wt/Wt | no | no | 115 |
| Decreased | Intermediate | Wt/Wt | yes | no | 8 |
| | | Wt/*4 | no | no | 32 |
| | Poor | unknown | no | yes | 1 |
| | | *4/*4 | unknown | unknown | 2 |
| | | *4/*4 | no | no | 11 |
| | | Wt/*4 | no | yes | 1 |
| | | Wt/Wt | no | yes | 1 |
| | Unclassified | unknown | yes | no | 2 |
| | | Wt/*4 | unknown | unknown | 7 |

One hundred and fifteen patients were extensive metabolizers based on genotype (Wt/Wt) and the absence of a CYP2D6 inhibitor. In contrast, 65 patients had evidence for “decreased” metabolism by genotype (n=53), or by the co-administration of a CYP2D6 inhibitor (n=13). One patient with the *4/Wt genotype was also prescribed a CYP2D6 inhibitor. Of the 65 patients with decreased metabolism, patients were subdivided into intermediate (n=40) or poor metabolizers (n=16) based on genotype (*4/wt vs. *4/*4) and the potency of the inhibitor prescribed (moderate vs. potent). Nine patients with “decreased” metabolism could not be classified further because of lack of medication history [*4/Wt (n=7)] or because genotype was unknown but there was documentation of a moderate CYP2D6 inhibitor (n=2). The characteristics of the 180 patients included in this study are listed in Table 2 and were similar to the eligible patients from the tamoxifen arm of 89-30-52 in which metabolizer status could not be determined (n=76).

TABLE 2
Patient characteristics comparing those with known versus unknown
CYP2D6 metabolizer status

| | Metabolizer status | Metabolizer status |
|---|---|---|
| Patient Characteristics | known (n = 80) | unknown (n = 76) |
| Median Age | 68 | 68 |
| (range) | (42-87) | (47-82) |
| Race | | |
| Caucasian | 95% | 84% |
| Afro-American | 1% | 4% |
| Native American | 1% | 0% |
| unknown | 3% | 12% |
| Operative Procedure | | |
| Mastectomy | 83% | 79% |
| Breast conserving | 17% | 21% |
| Tumor size | | |
| <3 cm | 78% | 82% |
| ≧3 cm | 22% | 18% |
| Number of positive nodes | | |
| 0 | 64% | 59% |
| 1-3 | 25% | 25% |
| 4-9 | 7% | 12% |
| 10+ | 4% | 4% |
| Estrogen Receptor (at | | |
| randomization) | | |
| 10-49 fmols | 22% | 16% |
| ≧50 fmols | 66% | 69% |
| positive | 13% | 16% |
| HER-2 | | |
| 0 | 11% | 8% |
| 1 | 32% | 22% |
| 2 | 33% | 16% |
| 3 | 19% | 8% |
| unknown | 5% | 46% |
| Nottingham tumor Grade | | |
| Grade 1 | 24% | 16% |
| Grade 2 | 54% | 28% |
| Grade 3 | 15% | 12% |
| Unknown | 7% | 44% |

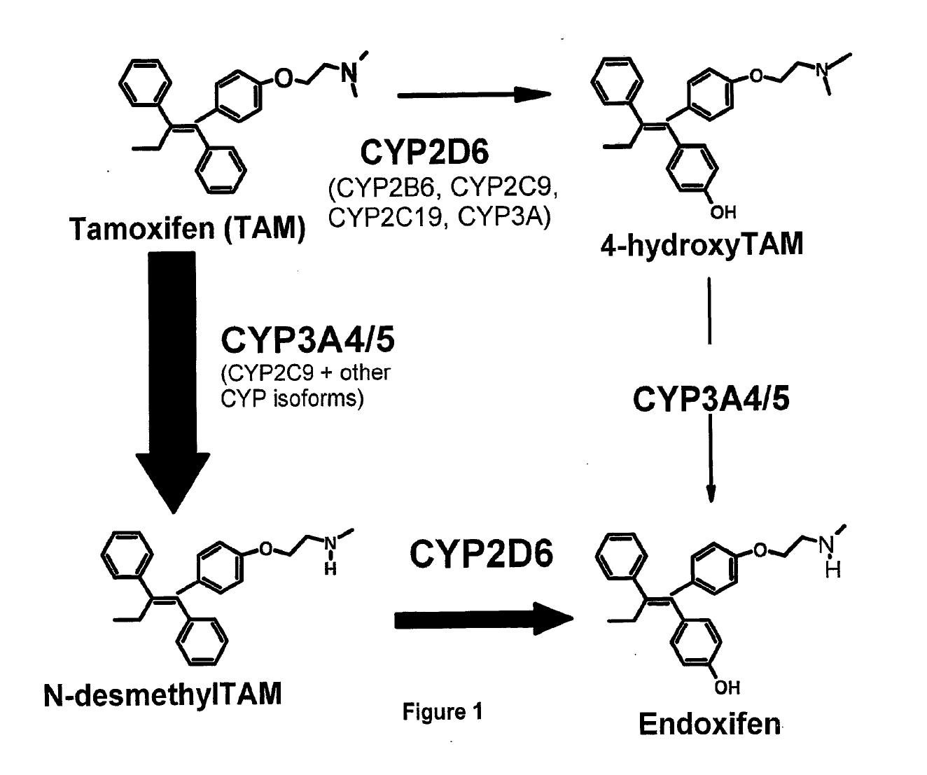

Clinical Outcome by CYP2D6 metabolism. Clinical benefit was significantly decreased for women with decreased CYP2D6 metabolism relative to women with extensive metabolism. Patients with decreased metabolism had significantly shorter TTBR (log-rank p=0.015), RFS (log-rank p=0.007), and DFS (log-rank p=0.009), and tended to have worse OS (log-rank p=0.082) compared with patients with extensive CYP2D6 metabolism (FIG. 1).

Cox proportional hazard modeling demonstrated that tumor size greater than 3 cm (yes vs. no) and positive nodes (yes vs. no) were significantly associated with TTBR, RFS, DFS, and OS. When tumor size and nodal status were taken into account, patients with decreased metabolism had significantly shorter TTBR (p=0.034; adj HR=1.91; 95% CI: 1.05-3.45); RFS (p=0.017; adj HR=1.74; 95% CI: 1.10-2.74); and DFS (p=0.027; adj HR=1.60; 95% CI: 1.06-2.43) relative to those with extensive metabolism. Overall survival did not differ significantly between these patient groups (p=0.223; adj HR=1.34; 95% CI: 0.83-2.16; Table 3).

TABLE 3
Hazard ratios (and corresponding 95% confidence intervals) of
multivariate Cox modeling comparing extensive versus decreased
CYP2D6 metabolism for time to breast cancer recurrence (TTBR),
relapse-free survival (RFS), and disease-free survival (DFS)

| | | | CYP2D6 Metabolism |
|---|---|---|---|
| | Size | Node positive | (decreased vs. |
| End point | (≧3 cm vs. <3 cm) | (yes vs. no) | extensive) |
| TTBR | 2.71 | 1.79 | 1.91 |
| | (1.16-4.07) | (0.93-3.08) | (1.05-3.45) |
| RFS | 1.75 | 1.57 | 1.74 |
| | (1.06-2.88) | (1.00-2.48) | (1.10-2.74) |
| DFS | 1.94 | 1.71 | 1.60 |
| | (1.24-3.04) | (1.13-2.58) | (1.06-2.43) |

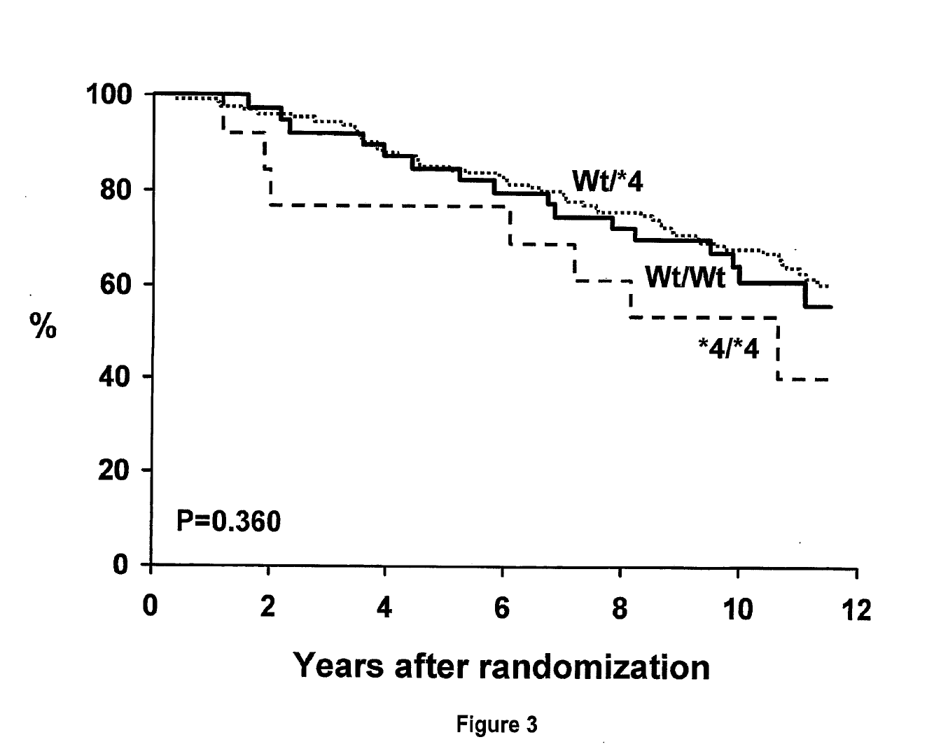

Clinical Outcome by Metabolizer Status. In those patients (n=173) in which metabolizer status could be determined (Table 1), a Cox model was used to assess whether clinical benefit was significantly decreased for intermediate or poor metabolizers relative to extensive metabolizers. Poor metabolizers had significantly shorter TTBR (p=0.007), RFS (p=0.005), and DFS (=0.008) and tended to have worse OS (p=0.077) compared to extensive metabolizers. Intermediate metabolizers did not have shorter TTBR (p=0.338) but tended to have worse RFS (p=0.075) and DFS (p=0.097) compared to extensive metabolizers (Table 4). The Kaplan Meier curves for TTBR, RFS, DFS, and OS by metabolizer class are presented in FIG. 2. The two year RFS rates by metabolizer status were 98%, 92%, and 68% for extensive, intermediate, and poor metabolizers, respectively (FIG. 2b). Furthermore, comparing instantaneous hazard rates for RFS by metabolizer status (FIG. 3) demonstrated an immediate broad peak in the RFS hazard rate for patients with decreased metabolism. In contrast, the hazard rate in patients with extensive metabolism was reduced and did not peak until nearly the fourth year. These findings suggest that the peak in the hazard rate for recurrence seen following the initiation of tamoxifen may be due to a subset of patients unable to fully activate tamoxifen.

TABLE 4
Hazard ratios (and corresponding 95% confidence intervals) of
univariate Cox modeling of time to breast cancer recurrence (TTBR),
relapse-free survival (RFS), disease-free survival (DFS), and
overall survival (OS) by metabolizer status

| | Estimated unadjusted hazard | |
|---|---|---|
| | ratio relative to extensive | |
| | metabolizers | |
| | (corresponding 95% | |
| Outcome | confidence interval) | p-value |
| Time to Breast recurrence | | |
| Poor Metabolizer | 3.2 (1.37-7.55) | 0.007 |
| Intermediate Metabolizer | 1.4 (0.68-3.05) | 0.3375 |
| Relapse-free survival | | |
| Poor Metabolizer | 2.69 (1.34-5.37) | 0.005 |
| Intermediate Metabolizer | 1.63 (0.95-2.78) | 0.075 |
| Disease-free survival | | |
| Poor Metabolizer | 2.44 (1.27-4.69) | 0.008 |
| Intermediate Metabolizer | 1.52 (0.93-2.49) | 0.097 |
| Overall survival | | |
| Poor Metabolizer | 2.0 (0.92-4.17) | 0.077 |
| Intermediate Metabolizer | 1.40 (0.80-2.43) | 0.240 |

The results presented herein demonstrate that patients with impaired CYP2D6 metabolism have a nearly two fold higher risk of breast cancer recurrence, independent of standard prognostic markers. Further, when patients were classified according to the extent to which CYP2D6 enzyme activity was reduced, the effect of reduced metabolism appeared greatest in poor metabolizers, with a greater than three fold higher risk of recurrence (HR 3.12, p=0.007) compared to extensive metabolizers.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for assessing the likelihood of cancer relapse, wherein said method comprises determining whether or not a human breast cancer patient contains a CYP2D6*4/*4 genotype, wherein the presence of said genotype indicates that said patient is likely to experience cancer relapse, and wherein the absence of said genotype indicates that said patient is likely to experience a relapse-free survival or disease-free survival.

2. The method of claim 1, wherein said patient contains said CYP2D6*4/*4 genotype, and said method comprises classifying said patient as being likely to experience cancer relapse.

3. The method of claim 1, wherein said patient does not contain said CYP2D6*4/*4 genotype, and said method comprises classifying said patient as being likely to experience a relapse-free survival or disease-free survival.

4. The method of claim 1, wherein said determining step comprises using PCR.

5. The method of claim 1, wherein said determining step comprises obtaining nucleic acid from a tumor sample of said patient.

6. The method of claim 1, wherein said determining step comprises obtaining nucleic acid from a non-tumor sample of said patient.

7. The method of claim 1, wherein said patient is a tamoxifen-treated patient.

8. A method for assessing the likelihood of a breast cancer patient to have hot flashes when treated with tamoxifen, wherein said method comprises determining whether or not said breast cancer patient contains a CYP2D6*4/*4 genotype, wherein the presence of said genotype indicates that said patient is not likely to experience hot flashes when treated with tamoxifen, and wherein the absence of said genotype indicates that said patient is likely to experience hot flashes when treated with tamoxifen.

9. The method of claim 8, wherein said patient contains said CYP2D6*4/*4 genotype, and said method comprises classifying said patient as being not likely to experience hot flashes when treated with tamoxifen.

10. The method of claim 8, wherein said patient does not contain said CYP2D6*4/*4 genotype, and said method comprises classifying said patient as being likely to experience hot flashes when treated with tamoxifen.

11. The method of claim 8, wherein said determining step comprises using PCR.

12. The method of claim 8, wherein said determining step comprises obtaining nucleic acid from a tumor sample of said patient.

13. The method of claim 8, wherein said determining step comprises obtaining nucleic acid from a non-tumor sample of said patient.
